(19) United States
(12) Patent Application Publication
(10) Pub. No.: US 2012/0226311 A1
(43) Pub. Date: Sep. 6, 2012

(54) CONFIGURABLE BACK ORTHOSIS

(75) Inventors: Jean-François Normandin, Carignan (CA); Sylvain Guimond, Sainte-Anne-de-Sorel (CA)

(73) Assignee: BIOTONIX (2010) INC., Brossard, QC (CA)

(21) Appl. No.: 13/383,655

(22) PCT Filed: Jul. 6, 2010
(86) PCT No.: PCT/CA10/01071

§ 371 Date: May 22, 2012

Related U.S. Application Data
(60) Provisional application No. 61/213,764, filed on Jul. 13, 2009.

(51) Int. Cl. A61B 17/00

(52) U.S. Cl. 606/240

(57) ABSTRACT

There is disclosed a configurable back orthosis, comprising a backplane, a plurality of support elements positioned on the backplane, each support element being associated with a respective area of the human body and a plurality of position adjusters, at least one position adjuster being associated with each of the support elements. Each support element may be adjusted using the associated at least one position adjuster in order to counteract a postural deviation of an individual using the configureable back orthosis.

### CROSS-REFERENCE TO RELATED APPLICATIONS

This application claims the benefits of U.S. provisional patent application No. 61/213,764 filed on Jul. 13, 2009, which is herein incorporated by reference.

### TECHNICAL FIELD

The present invention relates to a configurable back orthosis.

### BACKGROUND

Posture is defined as the position of the limbs or the carriage of the body as a whole. Science has shown that good posture allows the body to function and perform optimally. On the other hand, bad posture causes muscular imbalances and pressures on the nerves and internal organs which can lead to musculoskeletal disorders, aches and pains, joint degeneration, and poor bodily functions.

Posture evaluation software has been developed to identify deviations from an optimal position. Following an automated analysis, the application provides a customized exercise program to assist in correcting these deviations. The main drawback with this methodology is patient compliance, which may reduce the program benefits if the exercises are not performed as defined, or insufficiently frequently.

Treatment tables for therapists such as chiropractors typically allow some adjustments for patients such as height, arm rest positions, etc. These adjustments are mainly used for patient comfort and have no direct incidence on the treatment aside from optimizing access for the therapist. Some devices do include patient attachment mechanisms for the patient's upper and lower body section, and apply traction forces that provide a decompression treatment for some specific pathology, but none address the manipulations required to affect a patient's posture.

Accordingly, there is a need for a solution accessible to everyone, regardless of their inclination towards exercise, which induces displacements and applies forces to a patient's body in order to induce postural corrections.

### SUMMARY

According to one aspect of the present invention, there is provided a configurable back orthosis, comprising:
a backplane;
a plurality of support elements positioned on the backplane, each support element being associated with a respective area of the human body; and
a plurality of position adjusters, at least one position adjuster being associated with each of the support elements;
   wherein each support element can be adjusted using the associated at least one position adjuster in order to counteract a postural deviation of an individual using the configureable back orthosis.

According to another aspect of the present invention, there is provided a postural adjustment system, comprising:

an configurable back orthosis as described above;
an imaging unit; and
a processing unit operatively connected to the configurable back orthosis and the imaging unit, the processing unit being so configured so as to:
   acquire at least one digital image of an individual from the imaging unit;
   process the at least one digital image;
   identify one or more postural deviations of the individual;
   generate adjustments variables for the plurality of position adjusters in accordance with the one or more postural deviations;
   providing commands to adjust the plurality of position adjusters in accordance with the adjustment variables in order to correct the one or more postural deviations of the individual.

### BRIEF DESCRIPTION OF THE FIGURES

Embodiments of the invention will be described by way of examples only with reference to the accompanying drawings, in which:

FIG. 1 is a perspective top view of an illustrative embodiment of the configurable back orthosis;

FIG. 2 is a perspective top view of an individual laying on the configurable back orthosis of FIG. 1;

FIG. 3 is perspective top view of the configurable back orthosis of FIG. 1 without cushions;

FIG. 4 is a bottom perspective view of the configurable back orthosis of FIG. 1 without cushions;

FIG. 5 is a side view of the configurable back orthosis of FIG. 1 without cushions;

FIGS. 6a to 6e are schematic views of different embodiments of the configurable back orthosis of FIG. 1 used with a postural diagnostic system;

FIG. 7 is a flow diagram of an example of a process showing the use of the configurable back orthosis of FIG. 1 with a postural diagnostic system; and

FIG. 8 is a flow diagram of a postural analysis and adjustment variables generation sub-process used by the process of FIG. 7.

### DETAILED DESCRIPTION

A non-restrictive illustrative embodiment of the present invention relates to a configurable back orthosis which helps correct postural deviations of an individual in a passive way.

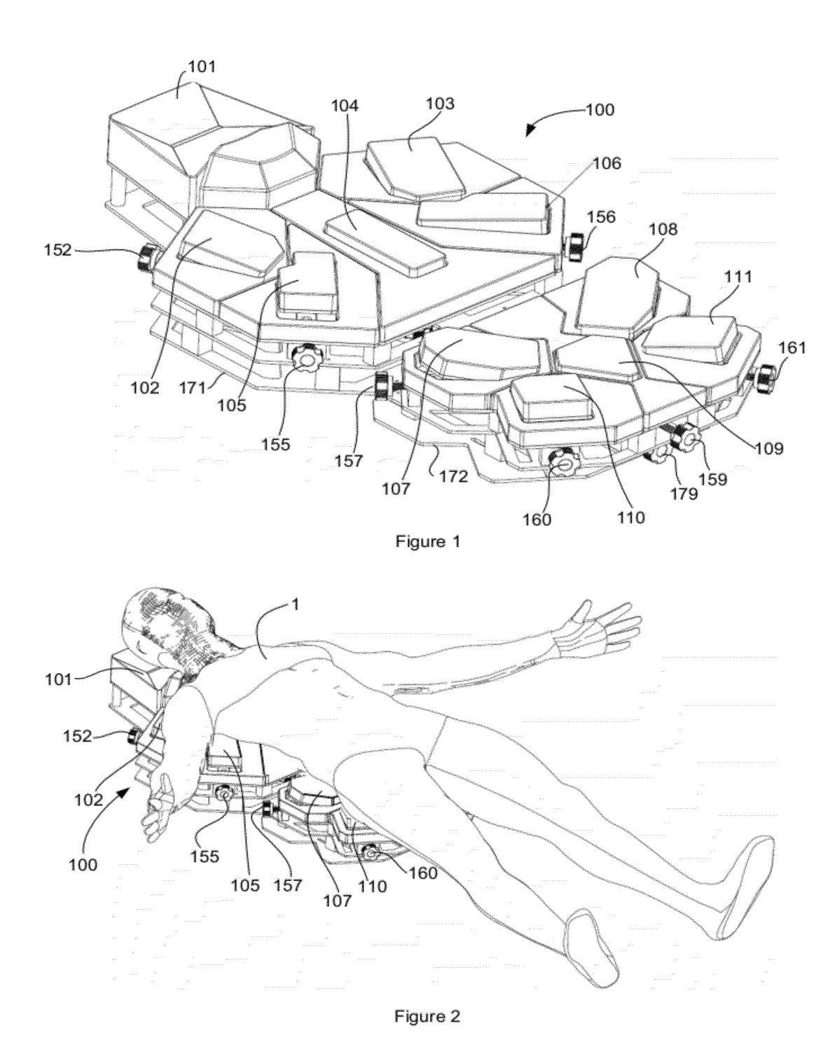

Referring to FIG. 1, the illustrative embodiment of the configurable back orthosis 100 comprises support elements in the form of, for example, eleven cushions 101 to 111 placed at associated strategic areas of the back. The cushions 101 to 111 correspond, respectively, to the following associated strategic areas: cervical (101), right shoulder (102), left shoulder (103), thoracic (104), right sub-scapular (105), left sub-scapular (106), right suprailiac (107), left suprailiac (108), sacrum (109), right buttock (110) and left buttock (111). The cushions 101 to 111 may be made, for example, of a polyurethane foam or similar material.

The cushions 101 to 111 can be adjusted in height and/or inclination according to the desired effect on an individual's posture to counteract the postural deviations identified by a health professional or postural diagnostic system. Referring to FIG. 2, once the various cushions 101 to 111 have been adjusted, the individual 1 lays on its back on top of the configurable back orthosis 100 in order to correct its posture. As will be shown later on, the configurable back orthosis 100 can also be adjusted in length and width to fit an individual's back regardless of its size. The configurable back orthosis 100 can also be provided with a fixed or extensible/retractable foot rest in order to provide for an optimal positioning of the individual. Alternatively, the individual 1 may lay on its back on top of the configurable back orthosis 100 while it is still in a neutral position and then the various cushions 101 to 111 are adjusted.

It is to be understood that the various adjustments of the configurable back orthosis 100, the frequency of its use as well as the length of use may vary from one individual to another according to a given diagnosis and practitioner follow-up.

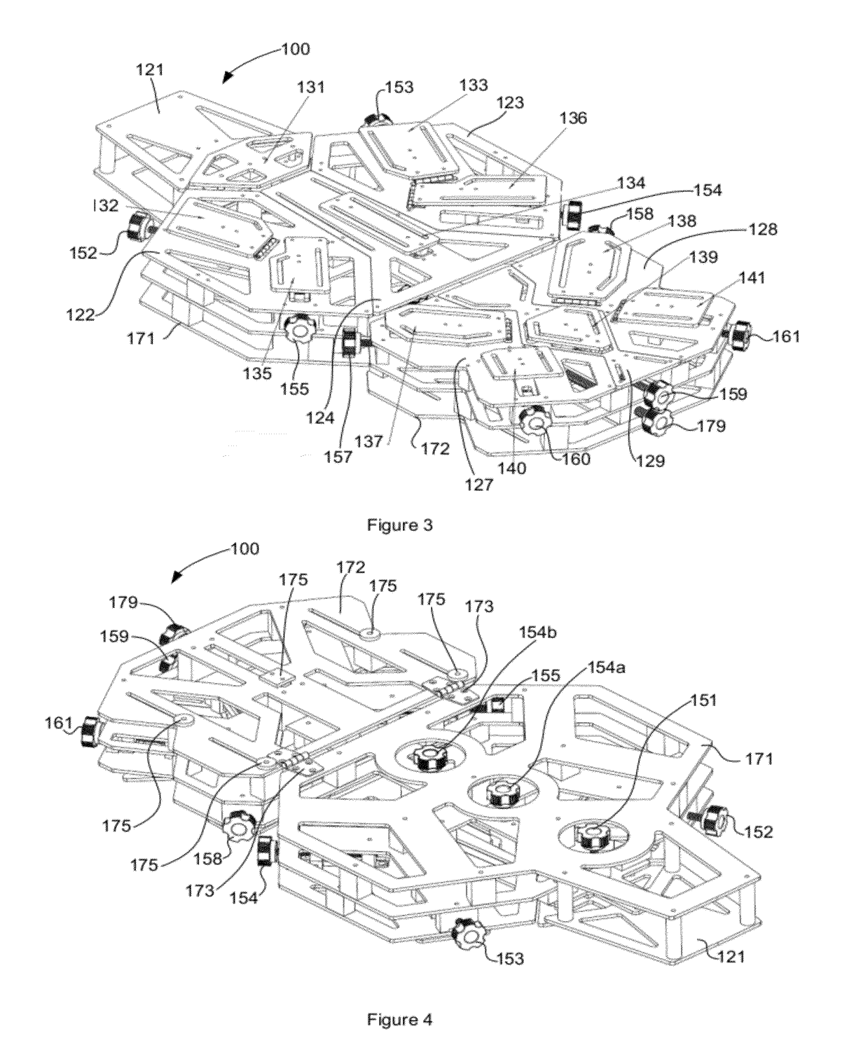

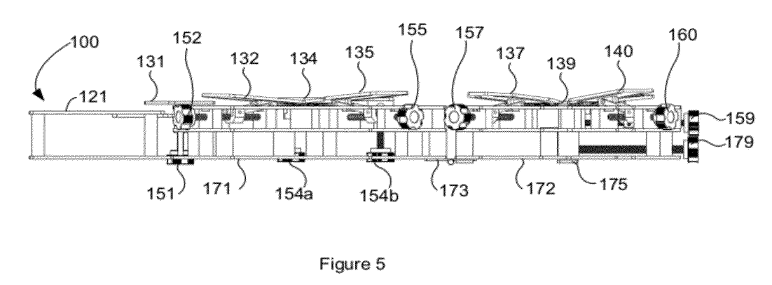

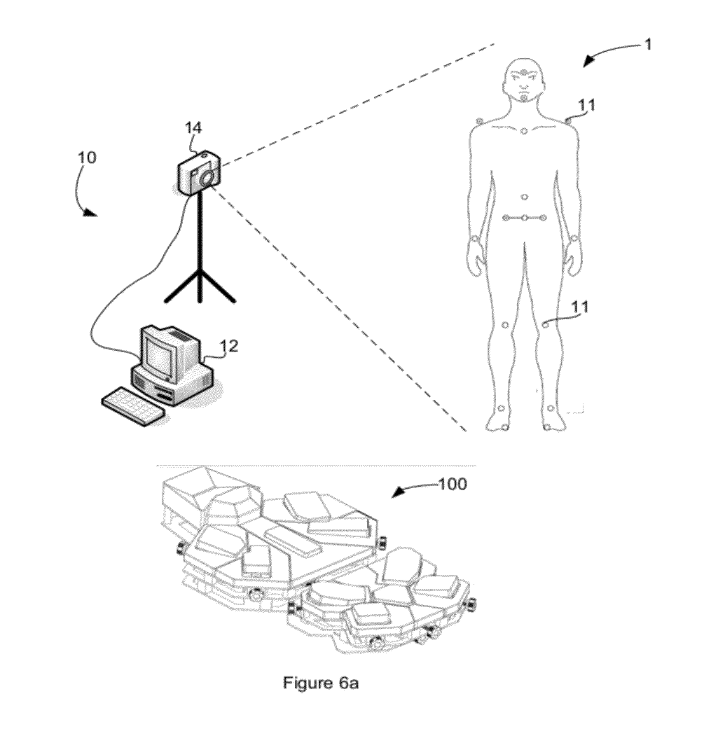

Referring now to FIGS. 3 to 5, there is shown the configurable back orthosis 100 without the cushions 101 to 111 (see FIG. 1). The body of the configurable back orthosis 100 is composed of seven support structures 121, 122, 123, 124, 127, 128 and 129, which may be operatively interconnected so as to allow the adjustment of the length and width of the configurable back orthosis 100. It is to be understood, however, that in alternative embodiments the body of the configurable back orthosis 100 may be composed of a different number of support structures, for example a single unitary support structure.

In the illustrative embodiment, as best seen in FIG. 4, the upper body support structures 121, 122, 123 and 124 are engaged to an upper body backplane 171 while the lower body support structures 127, 128 and 129 are engaged to a lower body backplane 172, the upper 171 and lower 172 body backplanes being pivotally connected together via hinges 173.

The purpose of the hinges 173 is to allow the folding of the configurable back orthosis 100 in order to facilitate its transport. Accordingly, it is to be understood that the hinges 173 may be omitted, in which case the upper 171 and lower 172 body backplanes may be combined into a single unitary backplane.

Referring still to FIG. 4, the lower body support structures 127, 128 and 129 are movably engaged to the lower body backplane 172 using guide elements 175. The length of the configurable back orthosis 100 may thus be adjusted by linearly moving the lower body support structures 127, 128 and 129 using a manual or automated actuator. In the illustrated embodiment, the actuator 179 is in the form of a set screw which may be manually adjusted to activate a follower operatively connected to the lower body support structures 127, 128 and 129. It is to be understood that additional actuators and guide elements may be used in order to provide relative moments between all or some of the support structures 121, 122, 123, 124, 127, 128 and 129.

Each support structure 121, 122, 123, 124, 127, 128 and 129 of the configurable back orthosis 100 body supports one or more position adjusters 131 to 141 each having an associated cushion 101 to 111, namely:
support structure 121 (neck and head) supports position adjuster 131 associated with cervical cushion 101;
support structure 122 (right upper back) supports position adjusters 132 and 135 associated with, respectively, the right shoulder 102 and right sub-scapular 105 cushions;
support structure 123 (left upper back) supports position adjusters 133 and 136 associated with, respectively, the left shoulder 103 and left sub-scapular 106 cushions;
support structure 124 (middle upper back) supports position adjuster 134 associated with the thoracic cushion 104;
support structure 127 (right lower back) supports position adjusters 137 and 140 associated with, respectively, the right suprailiac 107 and right buttock 110 cushions;
support structure 128 (left lower back) supports position adjusters 138 and 141 associated with, respectively, the left suprailiac 108 and left buttock 111 cushions; and
support structure 129 (middle lower back) supports position adjuster 139 associated with the sacrum cushion 109.

Each position adjuster 131 to 141 may be adjusted in height and/or inclination using one or more manual or automated actuator. In the illustrated embodiment of FIGS. 3 to 5, the actuators 151 to 161 are in the form of set screws which may be manually adjusted to either activate a follower operatively connected to a plate having a hinge at one end (e.g. actuators 152, 153 and 155 to 161) thus varying its inclination, or operatively connected directly to a plate in order to vary its height (e.g. actuators 151, 154a and 154b). In an alternative embodiment, the position adjusters 131 to 141 may be, for example, inflatable bladders incorporated into corresponding cushions 101 to 111.

Each position adjuster 131 to 141 may further include various position indicators or markers (not shown) corresponding to various heights, angles or other identifier in order to provide quantifiable settings.

In an alternative embodiment, the cushions 101 to 111 or position adjuster 131 to 141 may include heating elements and/or vibrators that may be activated in order to provide muscle relaxation.

In another alternative embodiment, the configurable back orthosis 100 may be provided with one or more belts having at least one surface electromyographic stimulator in order to stimulate the individual's 1 abdominals.

In a further alternative embodiment, the configurable back orthosis 100 may be provided with a primary respiratory movement (PRM) mechanism. The PRM is a movement present throughout the body and over which individuals have no control (i.e. it is an involuntary movement). The PRM is characterized by light movement of the bones in the skull and sacrum, the membrane system (visceral) and the central nervous systems cerebrospinal fluid. The PRM is felt as the expansion and contraction of the head and body as if the whole body is “breathing”. The cycle of the PRM is a complete expansion and contraction that occur 8 to 14 times per minute. The PRM is an indication of the level of vitality of an individual and helps the body's natural power of self-correction. If an individual has been ill or has suffered trauma, the rate and amplitude may be much lower than normal, or it may absent all together.

In order to reproduce the PRM, the PRM mechanism may comprise, for example, an automated actuator that repetitively expends the distance between the cervical 101 and sacrum 109 cushions and then contracts it following, for example, 8 to 14 expansion and contraction cycles per minute. The changed sensation that a user of the configurable back orthosis 100 may feel during and immediately after treatment may last for hours, a day or a week, but the deeper physiological effects will continue for a considerable time.

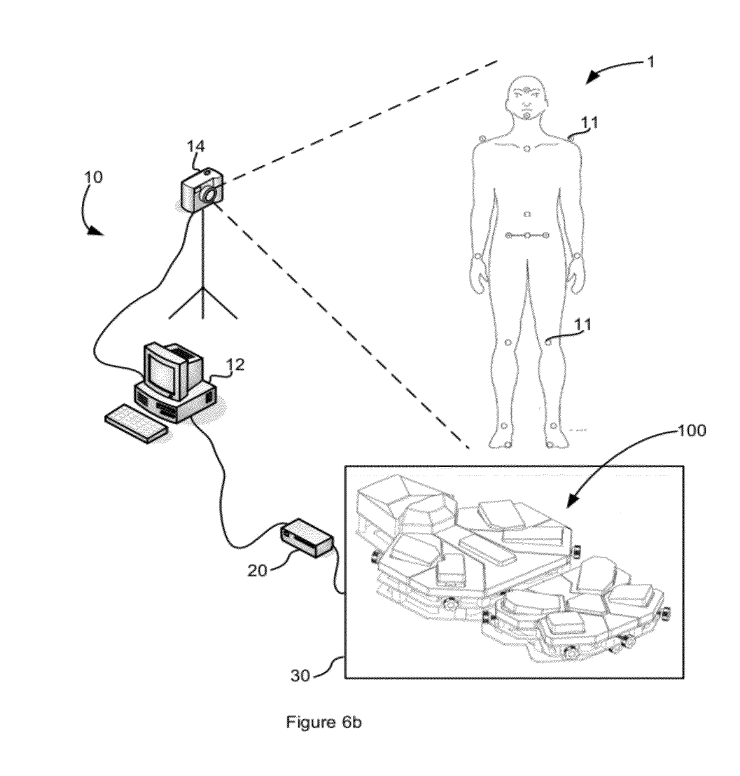

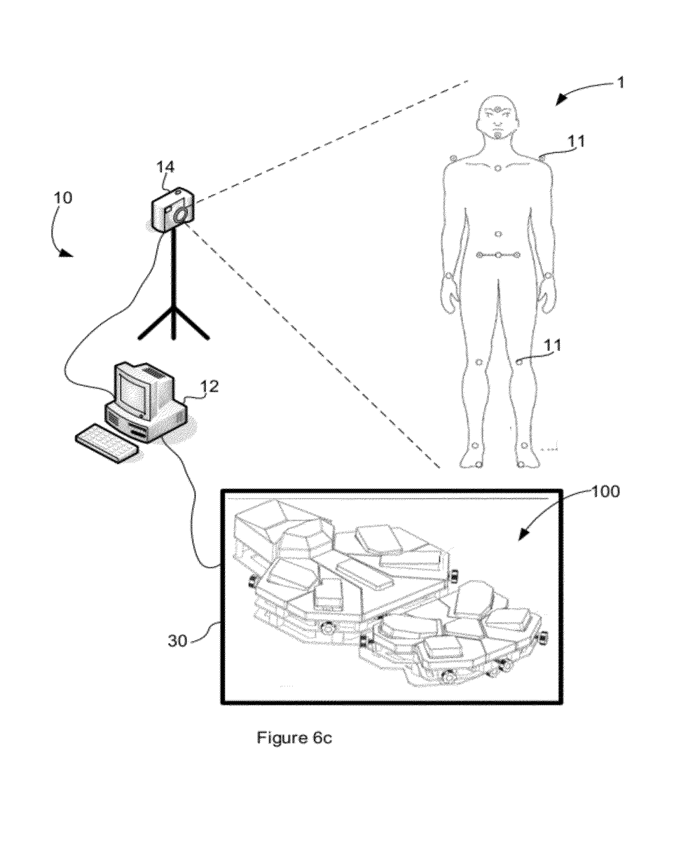

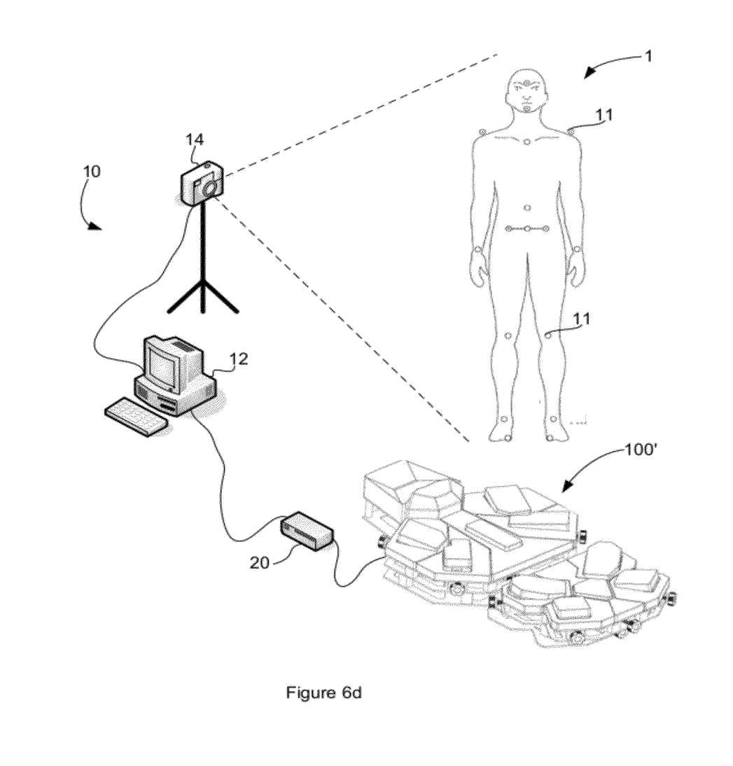

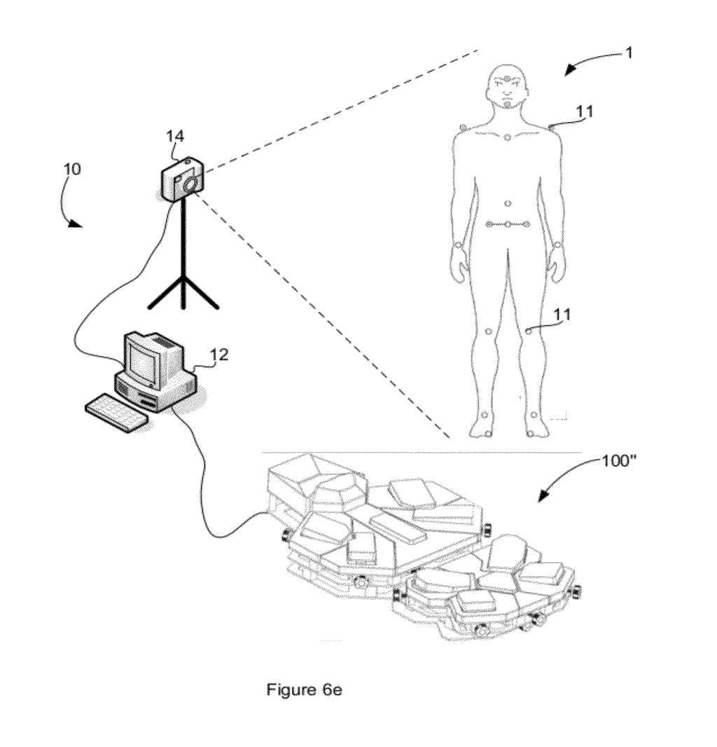

Referring now to FIG. 6a, the configurable back orthosis 100 may be used in conjunction with a postural diagnostic system 10 in order to provide a postural adjustment system. The postural diagnostic system 10 generally comprises a processing unit 12 such as, for example, a computer and an imagining unit 14 such as, for example, a digital camera or scanner. One or more digital images of an individual 1 are acquired by the imagining unit 14 and then treated by the processing unit 12 in order to establish a postural diagnostic of the individual 1. The processing unit 12 may use various techniques such as the analysis of the relative position of markers 11 positioned on the body of the individual 1 at specific anatomical locations. An example of an analysis technique that may be implemented by the processing unit 12 is described in U.S. Pat. No. 6,514,219 entitled “SYSTEM AND METHOD FOR AUTOMATED BIOMECHANICAL ANALYSIS AND THE DETECTION AND CORRECTION OF POSTURAL DEVIATION” by Guimond et al. It is to be understood that other analysis techniques may also be used.

Once a diagnosis is established, it may be used together with a correspondence table so as to determine the proper adjustments to be made for one or more of the position adjusters 131 to 141 in order to correct the posture of the individual 1. Alternatively, algorithms or computations may be substituted for the correspondence table.

Table 1 shows an example of a correspondence table identifying the position adjusters that are to be adjusted in order to counteract postural deviations identified by the postural diagnostic system 10. It is to be understood that the specific adjustments may depend on the severity of the diagnosed postural deviation and other parameters such as the individual's age, physical condition, etc.

TABLE 1
Correspondence between postural deviations and position adjusters

| Deviation | Position Adjuster(s) |
|---|---|
| Head Forward Protrusion | 101 |
| Neck Flexion | 101 |
| Neck Extension | 101 |
| Right Shoulder Elevation | 102, 106 |
| Left Shoulder Elevation | 103, 105 |
| Shoulder Protraction | 104 |
| Kyphosis | 104 |
| Trunk Positive Rotation | 103, 106 |
| Trunk Negative Rotation | 102, 105 |
| Right Pelvis Elevation | 107, 111 |
| Left Pelvis Elevation | 108, 110 |
| Forward Pelvic Tilt | 109 |
| Backward Pelvic Tilt | 109 |
| Pelvic Positive Rotation | 108, 111 |
| Pelvic Negative Rotation | 107, 110 |

It is to be understood that the correspondence table may contain other related data such as, for example, the desired amplitude of the adjustment, depending on the severity of the postural deviation, as well as any considerations for multiple simultaneous postural deviations, historic of the individual, etc.

In an alternative embodiment shown in FIG. 6b, a configuration determination unit 20 and a configuration interface 30 may be used with the postural diagnostic system 10 in order to automatically configure the configurable back orthosis 100 in accordance with the diagnosis data obtained from the processing unit 12. The configuration determination unit 20 implements a correspondence table and other related data from the processing unit 12 so as to provide the proper adjustments to be made for one or more of the position adjusters 131 to 141 to the configuration interface 30 which is operatively connected to the configurable back orthosis 100. The configuration interface 30 may be provided with interface ports corresponding to each of the position adjusters 131 to 141, the interface ports being designed so as to adjust the position adjusters 131 to 141 without requiring human intervention.

In another alternative embodiment, shown in FIG. 6c, the correspondence table and other related data may be implemented within the processing unit 12 so as to directly provide the proper adjustments to be made for one or more of the position adjusters 131 to 141 to the configuration interface 30 without requiring the use of the configuration determination unit 20 of FIG. 6b.

In a further alternative embodiment, shown in FIG. 6d, an automated configurable back orthosis 100′ may used with the postural diagnostic system 10 and the configuration determination unit 20, the automated configurable back orthosis 100′ having the control circuitry to adjust the automated actuators of each position adjuster 131 to 141.

In yet another alternative embodiment, shown in FIG. 6e, the correspondence table and other related data may be implemented within the automated configurable back orthosis 100″ so as to directly provide the proper adjustments to be made for one or more of the position adjusters 131 to 141 without requiring the use of the configuration determination unit 20 of FIG. 6d. Alternatively, the correspondence table and other related data may be implemented within the processing unit 12, as per FIG. 6c, with the automated configurable back orthosis 100″ simply implementing the provided adjustments.

It is also to be understood that the automated configurable back orthosis 100″ may be provided with a user interface allowing the control of the position adjusters 131 to 141 and, if applicable, the support structures 121, 122, 123, 124, 127, 128 and 129 and/or heating elements and/or vibrators and/or the PRM mechanism. The user interface may also allow the selection of various pre-programmed postural deviation treatments, save configurations, set treatment durations, etc. Alternatively, the user interface may furthermore allow programmation of the automated configurable back orthosis 100″.

It is to be understood that the configurable back orthosis 100 as well as the automated configurable back orthoses 100′ and 100″ may take other forms. For example, they may be incorporated within a mattress, a bed, a stretcher, a wheelchair, etc.

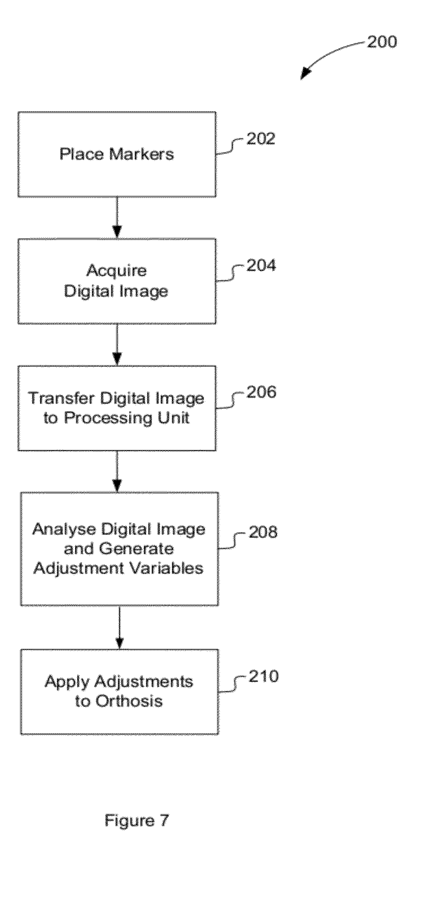

Referring now to FIG. 7, there is shown a flow diagram of an example of a process showing the use of the configurable back orthosis 100, or automated configurable back orthoses 100′, 100″, with a postural diagnostic system 10 such as the one described in U.S. Pat. No. 6,514,219. The steps of the process 200 are indicated by blocks 202 to 210, with references to FIGS. 6a to 6e.

The process 200 starts at block 202 where position markers 11 are positioned on the body of the individual 1 at specific anatomical locations.

At block 204, one or more digital images of the individual 1 are acquired by the imagining unit 14 and provided, at block 206, to the processing unit 12.

At block 208, the processing unit 12 executes a postural analysis and adjustment variables generation sub-process on the data of the one or more digital images in order to establish a postural diagnostic of the individual 1 and generate adjustment variables for the position adjusters 131 to 141 in order to treat one or more diagnosed postural deviations.

Then, at block 210, the position adjusters 131 to 141 are adjusted in accordance with the adjustment variables generated at block 208. Depending on the specifics of the postural diagnostic system 10, the position adjusters 131 to 141 may be adjusted manually (FIG. 6a), automatically via a configuration determination unit 20 along with a configuration interface 30 (FIG. 6b), automatically using a configuration interface 30 (FIG. 6c), automatically via a configuration determination unit 20 and an automated configurable back orthosis 100′ (FIG. 6d) or automatically via an automated configurable back orthosis 100″ (FIG. 6e).

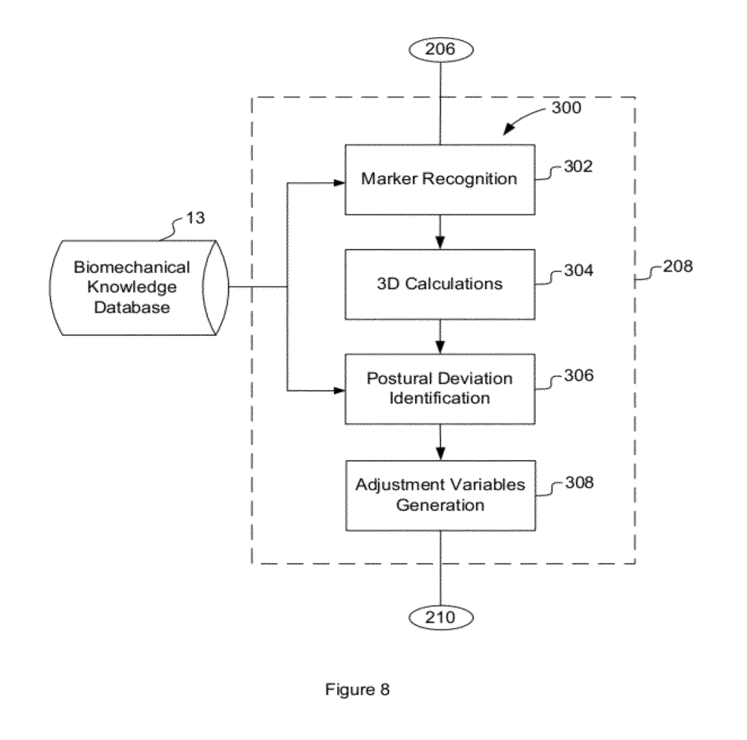

Referring now to FIG. 8, there is shown a flow diagram of an example of a postural analysis and adjustment variables generation sub-process 300 used at block 208 of process 200 (see FIG. 7). The steps of the sub-process 300 are indicated by blocks 302 to 308.

The sub-process 300 starts at block 302 where the position markers 11 are identified in the one or more digital images of the individual 1 using data from a biomechanical knowledge database 13 and their three-dimensional (3D) coordinates in space are determined. The biomechanical knowledge database 13 may be incorporated into the processing unit 12, provided as a separate database in the postural diagnostic system 10 or accessed remotely from a further system and/or database.

At block 304, various deviations and biomedical parameters are computed by comparing the 3D coordinates of the position markers 11, determined at block 302, with corresponding expected 3D coordinates in a healthy individual from the biomechanical knowledge database 13.

Then, at block 306, using the computed deviations and biomedical parameters, postural deviations are identified using the biomechanical knowledge database 13.

Finally, at block 308, using the identified postural deviations, adjustment variables for the position adjusters 131 to 141 are generated in order to correct the deviations. These adjustment variables may be generated, as previously mentioned, by the processing unit 12, and associated configuration determination unit 20 or by automated configurable back orthosis 100″.

Although the present invention has been described by way of particular embodiments and examples thereof, it should be noted that it will be apparent to persons skilled in the art that modifications may be applied to the present particular embodiments without departing from the scope of the present invention.

1. A configurable back orthosis, comprising:
a backplane;
a plurality of support elements positioned on the backplane, each support element being associated with a respective area of the human body; and
a plurality of position adjusters, at least one position adjuster being associated with each of the support elements;
wherein each support element can be adjusted using the associated at least one position adjuster in order to counteract a postural deviation of an individual using the configureable back orthosis.

2. The configurable back orthosis of claim 1, wherein at least one of the position adjusters is configured to adjust the height of its associated support element.

3. The configurable back orthosis of claim 1, wherein at least one of the position adjusters is configured to adjust the inclination of its associated support element.

4. The configurable back orthosis of claim 1, wherein the backplane is composed of an upper backplane pivotally connected to a lower backplane, therefore allowing folding of the configurable back orthosis.

5. The configurable back orthosis of claim 1, further comprising a plurality of support structures supported by the backplane, each support structure having associated support elements and position adjusters.

6. The configurable back orthosis of claim 5, wherein the plurality of support structures are operatively interconnected so as to allow the adjustment of the length of the configurable back orthosis.

7. The configurable back orthosis of claim 5, wherein the plurality of support structures are operatively interconnected so as to allow the adjustment of the width of the configurable back orthosis.

8. (canceled)

9. The configurable back orthosis of claim 5, wherein the support elements are associated with the cervical area, the right shoulder area, the right sub-capular area, the left shoulder area, the left sub-capular area, the thoracic area, the right suprailiac area, the right buttock area, the left suprailiac area, the left buttock area and the sacrum area.

10. (canceled)

11. (canceled)

12. The configurable back orthosis of claim 1, wherein the position adjusters include inflatable bladders.

13. The configurable back orthosis of claim 1, wherein at least one of the support elements includes a heating element.

14. The configurable back orthosis of claim 1, wherein at least one of the support elements includes a vibrator.

15. The configurable back orthosis of claim 1, further comprising a belt having at least one surface electromyographic stimulator in order to stimulate the individual's abdominals.

16. The configurable back orthosis of claim 1, further comprising a primary respiratory movement mechanism including an automated actuator operatively connecting the support elements associated with the cervical and the sacrum areas of the human body, the actuator being configured so as to repetitively expend and contract the distance between the support elements associated with the cervical and the sacrum areas of the human body.

17. The configurable back orthosis of claim 16, wherein the expending and contracting of the distance between the support elements associated with the cervical and the sacrum areas of the human body repeats from 8 to 14 times per minute.

18. The configurable back orthosis of claim 1, wherein the configurable back orthosis is incorporated into a mattress, a bed, a stretcher or a wheelchair.

19. A postural adjustment system, comprising:
an configurable back orthosis according to claim 1;
an imaging unit; and
a processing unit operatively connected to the configurable back orthosis and the imaging unit, the processing unit being so configured so as to:
acquire at least one digital image of an individual from the imaging unit;
process the at least one digital image;
identify one or more postural deviations of the individual;
generate adjustments variables for the plurality of position adjusters in accordance with the one or more postural deviations;
providing commands to adjust the plurality of position adjusters in accordance with the adjustment variables in order to correct the one or more postural deviations of the individual.

20. The postural adjustment system of claim 19, further comprising a correspondence table and wherein the adjustments to be made to the plurality of position adjusters a generated by applying the correspondence table to the one or more postural deviations.

21. The postural adjustment system of claim 19, further comprising a configuration interface for applying the adjustment commands provided by the processing unit to the plurality of position adjusters of the configurable back orthosis.

22. The postural adjustment system of claim 19, further comprising a plurality of markers adapted to be placed at specific anatomical locations on the body of the individual and a biomechanical database, the processing unit being further so configured so as to:
obtain 3D coordinates of the markers from the at least one digital image;
compare the obtained 3D coordinates with corresponding expected 3D coordinates in a healthy individual from the biomechanical database; and
compute biomechanical parameters from the 3D coordinates comparison;
wherein the one or more postural deviations are identified using the computed biomechanical parameters and the biomechanical database.
